(19) United States
(12) Patent Application Publication
(10) Pub. No.: US 2012/0029256 A1
(43) Pub. Date: Feb. 2, 2012

(54) PROCESS FOR THE CONVERSION OF LOWER ALKANES TO AROMATIC HYDROCARBONS

(75) Inventors: Yc-Mon Chen, Sugar Land, TX (US); Mahesh Venkataraman Iyer, Houston, TX (US); Karel Martin Kapoun, Sugar Land, TX (US); Ann Marie Lauritzen, Houston, TX (US); Ajay Madhav Madgavkar, Katy, TX (US)

(73) Assignee: SHELL INTERNATIONALE RESEARCH MAATSCHAPPIJ B.V., The Hague (NL)

(21) Appl. No.: 13/255,924

(22) PCT Filed: Mar. 10, 2010
(86) PCT No.: PCT/US2010/026778

§ 371 Date: Oct. 17, 2011

Related U.S. Application Data
(60) Provisional application No. 61/159,491, filed on Mar. 12, 2009.

(51) Int. Cl. C07C 15/00

(52) U.S. Cl. 585/407

(57) ABSTRACT

A process is provided for producing aromatic hydrocarbons which comprises: (a) contacting a lower alkane feed with a solid particulate aromatic hydrocarbon conversion catalyst in a fluidized bed reaction zone to produce aromatic hydrocarbons and other products, whereby the catalyst is at least partly deactivated by the formation of undesirable coke deposits, (b) continuously withdrawing a portion of the catalyst from the reaction zone, regenerating it in a regeneration zone and returning regenerated catalyst to the reaction zone, (c) maintaining the heat balance between the reaction zone and the regeneration zone by diluting the catalyst particles with particles of a catalytically inactive solid with about the same or improved specific heat and thermal conductivity relative to the catalyst, (d) separating aromatic hydrocarbons from the other products and unreacted lower alkanes, and (e) optionally recycling unreacted lower alkanes to the reaction zone.

This application claims priority to U.S. Provisional Application No. 61/159,491, filed on Mar. 12, 2009, which is herein incorporated by reference.

### FIELD OF THE INVENTION

The present invention relates to a process for producing aromatic hydrocarbons from lower alkanes. More specifically, the invention relates to a process for increasing the production of benzene from lower alkanes in a dehydroaromatization process.

### BACKGROUND OF THE INVENTION

There is a projected global shortage for benzene which is needed in the manufacture of key petrochemicals such as styrene, phenol, nylon and polyurethanes, among others. Generally, benzene and other aromatic hydrocarbons are obtained by separating a feedstock fraction which is rich in aromatic compounds, such as reformates produced through a catalytic reforming process and pyrolysis gasolines produced through a naphtha cracking process, from non-aromatic hydrocarbons using a solvent extraction process.

In an effort to meet growing world demand for key petrochemicals, various industrial and academic researchers have been working for several decades to develop catalysts and processes to make light aromatics, benzene, toluene, xylenes (BTX) from cost-advantaged, light paraffin (C₁-C₄) feeds. Catalysts devised for this application usually contain a crystalline aluminosilicate (zeolitic) material such as ZSM-5 and one or more metals such as Pt, Ga, Zn, Mo, etc. to provide a dehydrogenation function. Aromatization of ethane and other lower alkanes is thermodynamically favored at high temperature and low pressure without addition of hydrogen to the feed. Unfortunately, these process conditions are also favorable for rapid catalyst deactivation due to formation of undesirable surface coke deposits which block access to the active sites of the catalyst.

One approach to circumvent this rapid deactivation problem is to devise a lower alkane aromatization process featuring a fluidized catalyst bed in which catalyst particles cycle rapidly and continuously between a reaction zone where aromatization takes place and a regeneration zone where the accumulated coke is burned off the catalyst to restore activity. For example, U.S. Pat. No. 5,053,570 describes a fluid-bed process for converting lower paraffin mixtures to aromatics.

Due to the highly endothermic nature of the alkane aromatization reaction, there is a need to maintain a heat balance between the reaction and regeneration sections of the fluidized-bed system. This requirement can in principle be satisfied by maintaining a high inventory of solid catalyst particles in the system. However, high catalyst costs can make this approach prohibitively expensive, especially when one considers the high catalyst replenishment or makeup rate needed to compensate for the normal attrition and deactivation of catalyst particles during fluidized-bed operation.

### SUMMARY OF THE INVENTION

The invention relates to a fluidized-bed process for aromatization of lower alkanes utilizing an alkane aromatization catalyst diluted with a second, inert solid material. The present invention calls for meeting the need for heat balance, adequate heat transfer, and high solid circulation rate by diluting the catalyst particles with particles of a less expensive, catalytically inactive solid with similar or improved specific heat and thermal conductivity relative to the catalyst material.

A process is provided for producing aromatic hydrocarbons which comprises:

(a) contacting a lower alkane feed with a solid particulate aromatic hydrocarbon conversion catalyst in a fluidized bed reaction zone to produce aromatic hydrocarbons and other products, whereby the catalyst is at least partly deactivated by the formation of undesirable coke deposits,

(b) continuously withdrawing a portion of the catalyst from the reaction zone, regenerating it in a regeneration zone and returning regenerated catalyst to the reaction zone,

(c) maintaining the heat balance between the reaction zone and the regeneration zone by diluting the catalyst particles with particles of a catalytically inactive solid with about the same or improved specific heat relative to the catalyst, and preferably by maintaining a ratio of catalytically inactive solid circulation rate between the zones to catalyst particle circulation rate between the zones of about 1:6 to about 6:1, preferably about 0.4:1 to about 2.5:1,

(d) separating aromatic hydrocarbons from the other products and unreacted lower alkanes, and

(e) optionally recycling unreacted lower alkanes to the reaction zone.

In one embodiment of the present invention, the specific heat of the catalytically inactive solid is at least about 0.2 Btu/(lb-° R) (about 0.8 kJ/(kg-° K)). In another embodiment, the specific heat of the catalytically inactive solid is from about 0.2 to about 0.4 Btu/(lb-° R) (about 0.8 to about 1.7 kJ/(kg-° K)) at the temperature of operation.

“At the temperature of operation” relates to the changes that may occur in specific heat when the temperature is increased from ambient to the reaction temperature (for example, the specific heat of DENSTONE® 80 bed support media below is about 1.05 at ambient temperature and 1.18 in the range of the reaction temperature of this invention). The temperature of operation is generally about 200 to about 1000° C., preferably from about 300 to about 850° C., most preferably from about 575 to about 750° C.

### BRIEF DESCRIPTION OF THE DRAWINGS

FIG. 1 is a flow diagram which illustrates a process scheme for producing aromatics (benzene and higher aromatics) from lower alkanes by circulating excess solid catalyst particulates or a mixture of inert solid particulates and catalyst particulates as the heat transfer agent between the reaction and the regeneration zones.

### DETAILED DESCRIPTION OF THE INVENTION

The present invention is a process for producing aromatic hydrocarbons which comprises bringing a hydrocarbon feedstock generally containing at least about 50 percent by weight of lower alkanes and a catalyst composition suitable for promoting the reaction of lower alkanes to aromatic hydrocarbons such as benzene into contact at a temperature of about 200 to about 1000° C., preferably from about 300 to about 850° C., most preferably from about 575 to about 750° C. and a pressure of about 0.01 to about 0.5 MPa. The primary desired products of the process of this invention are benzene, toluene and xylene.

The hydrocarbons in the feedstock may include ethane, propane, butane, and/or C₅₊ alkanes or any combination thereof. Preferably, the majority of the feedstock is ethane and propane. The feedstock may contain in addition other open chain hydrocarbons containing between 3 and 8 carbon atoms as coreactants. Specific examples of such additional coreactants are propylene, isobutane, n-butenes and isobutene. The hydrocarbon feedstock preferably contains at least about 30 percent by weight of C₂₋₄ hydrocarbons, more preferably at least about 50 percent by weight.

This invention relates to a processing scheme for producing benzene (and other aromatics) from a mixed lower alkane stream which may contain C₂, C₃, C₄ and/or C₅₊ alkanes, for example an ethane/propane/butane-rich stream derived from natural gas, refinery or petrochemical streams including waste streams. Examples of potentially suitable feed streams include (but are not limited to) residual ethane and propane from natural gas (methane) purification, pure ethane, propane and butane streams (also known as Natural Gas Liquids) co-produced at a liquefied natural gas site, C₂-C₅ streams from associated gases co-produced with crude oil production, unreacted ethane “waste” streams from steam crackers, and the C₁-C₃ byproduct stream from naphtha reformers. The lower alkane feed may be deliberately diluted with relatively inert gases such as nitrogen and/or with various light hydrocarbons and/or with low levels of additives needed to improve catalyst performance.

The alkane aromatization reaction is highly endothermic and requires a great amount of heat. At high temperatures, the aromatization catalysts rapidly deactivate due to formation of undesirable surface coke deposits which block access to the active sites of the catalyst. Catalyst from the fluidized bed reaction zone in the process of the present invention may be rapidly and continuously cycled between the reaction zone and a regeneration zone where the accumulated coke is burned off of or otherwise removed from the catalyst to restore its activity. Thus, the process in the regeneration zone is exothermic and generates heat.

It is important that an equilibrium be established between the gain and loss of heat in the reaction system, i.e., a heat balance must be established. In the present invention, this is particularly important because of the endothermicity of the reaction section, the exothermicity of the regeneration section and the expensive heat exchange system that would be required at both the reaction and regeneration section if this heat balance is not established.

A heat balance could be established by maintaining a high inventory of solid catalyst particles in the reaction system. This would work because (a) the excess amount of catalyst solids may absorb the heat during coke burn in the regeneration section preventing the temperature to rise to levels that could be detrimental to the catalyst (b) the excess hot solids may also provide all the heat necessary for the endothermic reactions. However, the aromatization catalysts are expensive and taking this approach would dramatically increase the cost of the process, especially considering the high catalyst replenishment or make up rate needed to compensate for the normal attrition and deactivation of catalyst particles during fluidized bed operation.

The present invention provides a solution to the problem of establishing heat balance in the reaction system. Instead of using a large excess of catalyst particles, the desired amount of catalyst necessary for the size of the reactor and the amount of feed may be utilized. The catalyst particles may then diluted by the addition of particles of a catalytically inactive solid which will assist in transferring heat from the regeneration zone to the reaction zone without using heat exchange systems for both zones.

The ratio of the circulation rate (mass per unit of time) of the inert particles to the circulation rate of the catalyst particles (mass per unit of time) may be at least about 1:6 because less inert material than that would provide very little value in terms of enhanced heat transfer. Generally, the circulation rate ratio may be as much as about 6:1. No more than this may generally be used because the amount of catalyst may be inadequate for the reaction. Preferably, the ratio may be from about 0.4:1 to about 2.5:1 to achieve good heat transfer and sufficient reaction.

Best results will be achieved when the catalytically inactive solid has about the same or improved heat transfer properties relative to the catalyst. Specific heat capacity (also known simply as specific heat) is an important characteristic for the choice of the catalytically inactive solid.

It is preferred that the specific heat capacity of the catalytically inactive solid be about the same as that of the catalyst itself or improved (greater). Preferably, the specific heat of the catalytically inactive solid particles may be at least about 0.2 Btu/(lb-° R) (0.8 kJ/(kg-° K)) at the temperature of operation, more preferably from about 0.2 to about 0.4 Btu/(lb-° R) (from about 0.8 to about 1.7 kJ/(kg-° K)), most preferably from about 0.25 to about 0.35 Btu/lb/oR Btu/(lb-° R) (from about 1.04 to about 1.5 kJ/(kg-° K) because higher specific heats result in lower amount of solids in the system: either circulation, or inventory. Also, the specific heat ranges are preferred because they are close to that of the supported catalyst used in the invention.

Improved results may be achieved if the thermal characteristics of the catalytically inactive solid are improved in relation to those of the catalyst.

The catalytically inactive solid may be selected from alumina, silica, titania, clays, alkali oxides, alkaline earth oxides, bakelite, pyrex glass, limestone, gypsum, silicon carbide, and other refractory materials known to the practitioners of art and/or combinations thereof. Fixed bed support media such as DENSTONE® bed support media may be used in the present invention. For example, DENSTONE® 80 bed support media has a specific heat capacity of 0.28 Btu/(lb-° R) (1.18 kJ/(kg-° K)) at the temperature of operation. A typical aromatization catalyst such as the one described in U.S. Provisional Application 61/029,481 discussed below has a specific heat capacity of 0.28 Btu/(lb-° R) (1.17 kJ/(kg-° K)) at the temperature of operation. These two materials would match up well for use in the present invention. Other catalytically inactive solids which should also work well are shown in Table 1 below with their specific heats (Cₚ).

TABLE 1

| | CP @ 600-700 C. | |
|---|---|---|
| Material | Cp (Btu/lb/degR) | Cp (kJ/kg/degK) |
| Denstone 80 | 0.283 | 1.18 |
| Quartz | 0.28 | 1.17 |
| concrete | 0.22 | 0.92 |
| silicate glass | 0.26 | 1.09 |
| Limestone | 0.285 | 1.19 |
| Silcia | 0.31 | 1.30 |
| Alumina | 0.285 | 1.19 |
| gypsum | 0.275 | 1.15 |
| CaO | 0.225 | 0.94 |
| Titania | 0.223 | 0.93 |
| MgO | 0.31 | 1.30 |
| K2O | 0.275 | 1.15 |
| Na2O | 0.355 | 1.49 |

The particle size of the inert material may vary depending upon the type of reactor used. For example, Denstone 80® ⅛ inch particles may be too big for fluid bed operation. A smaller size inert material particle may be needed. Generally, the particle size of the inert material may be in the same range as the particle size of the catalyst particles.

Any one of a variety of catalysts may be used to promote the reaction of lower alkanes to aromatic hydrocarbons. One such catalyst is described in U.S. Pat. No. 4,899,006 which is herein incorporated by reference in its entirety. The catalyst composition described therein comprises an aluminosilicate having gallium deposited thereon and/or an aluminosilicate in which cations have been exchanged with gallium ions. The molar ratio of silica to alumina is at least 5:1.

Another catalyst which may be used in the process of the present invention is described in EP 0 244 162. This catalyst comprises the catalyst described in the preceding paragraph and a Group VIII metal selected from rhodium and platinum. The aluminosilicates are said to preferably be MFI or MEL type structures and may be ZSM-5, ZSM-8, ZSM-11, ZSM-12 or ZSM-35.

Other catalysts which may be used in the process of the present invention are described in U.S. Pat. No. 7,186,871 and U.S. Pat. No. 7,186,872, both of which are herein incorporated by reference in their entirety. The first of these patents describes a platinum containing ZSM-5 crystalline zeolite synthesized by preparing the zeolite containing the aluminum and silicon in the framework, depositing platinum on the zeolite and calcining the zeolite. The second patent describes such a catalyst which contains gallium in the framework and is essentially aluminum-free.

Additional catalysts which may be used in the process of the present invention include those described in U.S. Pat. No. 5,227,557, hereby incorporated by reference in its entirety. These catalysts contain an MFI zeolite plus at least one noble metal from the platinum family and at least one additional metal chosen from the group consisting of tin, germanium, lead, and indium.

One preferred catalyst for use in this invention is described in U.S. Provisional Application No. 61/029,481, filed Feb. 18, 2008 entitled “Process for the Conversion of Ethane to Aromatic Hydrocarbons.” This application is hereby incorporated by reference in its entirety. This application describes a catalyst comprising: (1) about 0.005 to about 0.1% wt (% by weight) platinum, basis the metal, preferably about 0.01 to about 0.05% wt, (2) an amount of an attenuating metal selected from the group consisting of tin, lead, and germanium, which is no more than 0.02% wt less than the amount of platinum, preferably not more than about 0.2% wt of the catalyst, basis the metal; (3) about 10 to about 99.9% wt of an aluminosilicate, preferably a zeolite, basis the aluminosilicate, preferably about 30 to about 99.9% wt, preferably selected from the group consisting of ZSM-5, ZSM-11, ZSM-12, ZSM-23, or ZSM-35, preferably converted to the H+ form, preferably having a SiO₂/Al₂O₃ molar ratio of from about 20:1 to about 80:1, and (4) a binder, preferably selected from silica, alumina and mixtures thereof.

Another preferred catalyst for use in this invention is described in U.S. Provisional Application No. 61/029,939, filed Feb. 20, 2008 entitled “Process for the Conversion of Ethane to Aromatic Hydrocarbons.” This application is hereby incorporated by reference in its entirety. The application describes a catalyst comprising: (1) about 0.005 to about 0.1% wt (% by weight) platinum, basis the metal, preferably about 0.01 to about 0.06% wt, most preferably about 0.01 to about 0.05% wt, (2) an amount of iron which is equal to or greater than the amount of the platinum but not more than about 0.50% wt of the catalyst, preferably not more than about 0.20% wt of the catalyst, most preferably not more than about 0.10% wt of the catalyst, basis the metal; (3) about 10 to about 99.9% wt of an aluminosilicate, preferably a zeolite, basis the aluminosilicate, preferably about 30 to about 99.9% wt, preferably selected from the group consisting of ZSM-5, ZSM-11, ZSM-12, ZSM-23, or ZSM-35, preferably converted to the H+ form, preferably having a SiO₂/Al₂O₃ molar ratio of from about 20:1 to about 80:1, and (4) a binder, preferably selected from silica, alumina and mixtures thereof.

Another preferred catalyst for use in this invention is described in U.S. Provisional Application No. 61/029,478, filed Feb. 18, 2008 entitled “Process for the Conversion of Ethane to Aromatic Hydrocarbons.” This application is hereby incorporated by reference in its entirety. This application describes a catalyst comprising: (1) about 0.005 to about 0.1 wt % (% by weight) platinum, basis the metal, preferably about 0.01 to about 0.05% wt, most preferably about 0.02 to about 0.05% wt, (2) an amount of gallium which is equal to or greater than the amount of the platinum, preferably no more than about 1 wt %, most preferably no more than about 0.5 wt %; (3) about 10 to about 99.9 wt % of an aluminosilicate, preferably a zeolite, basis the aluminosilicate, preferably about 30 to about 99.9 wt %, preferably selected from the group consisting of ZSM-5, ZSM-11, ZSM-12, ZSM-23, or ZSM-35, preferably converted to the H+ form, preferably having a SiO₂/Al₂O₃ molar ratio of from about 20:1 to about 80:1, and (4) a binder, preferably selected from silica, alumina and mixtures thereof.

A hydrodealkylation reaction, which involves the reaction of toluene, xylenes, ethylbenzene, and higher aromatics with hydrogen to strip alkyl groups from the aromatic ring, may be incorporated to produce additional benzene and light ends including methane and ethane which are separated from the benzene. This step substantially increases the overall yield of benzene and thus is highly advantageous.

Both thermal and catalytic hydrodealkylation processes are known in the art. Thermal dealkylation may be carried out as described in U.S. Pat. No. 4,806,700, which is herein incorporated by reference in its entirety. Hydrodealkylation operation temperatures in the described thermal process may range from about 500 to about 800° C. at the inlet to the hydrodealkylation reactor. The pressure may range from about 2000 kPa to about 7000 kPa. A liquid hourly space velocity in the range of about 0.5 to about 5.0 based upon available internal volume of the reaction vessel may be utilized. Due to the exothermic nature of the reaction, it is often required to perform the reaction in two or more stages with intermediate cooling or quenching of the reactants. Two or three or more reaction vessels may therefore be used in series. The cooling may be achieved by indirect heat exchange or interstage cooling. When two reaction vessels are employed in the hydrodealkylation zone, it is preferred that the first reaction vessel be essentially devoid of any internal structure and that the second vessel contain sufficient internal structure to promote plug flow of the reactants through a portion of the vessel.

Alternatively, the hydrodealkylation zone may contain a bed of a solid catalyst such as the catalyst described in U.S. Pat. No. 3,751,503, which is herein incorporated by reference in its entirety. Another possible catalytic hydrodealkylation process is described in U.S. Pat. No. 6,635,792, which is herein incorporated by reference in its entirety. This patent describes a hydrodealkylation process carried out over a zeolite-containing catalyst which also contains platinum and tin or lead. The process is preferentially performed at temperatures ranging from about 250° C. to about 600° C., pressures ranging from about 0.5 MPa to about 5.0 MPa, liquid hydrocarbon feed rates from about 0.5 to about 10 hr-1 weight hourly space velocity, and molar hydrogen/hydrocarbon feedstock ratios ranging from about 0.5 to about 10.

### EXAMPLES

The examples provided below are intended to illustrate but not limit the scope of the invention.

### Example 1

The details of the preparation methods, fixed-bed lab-scale testing procedures, and comparative initial performance results obtained under ethane aromatization conditions with a Pt/Ga catalyst made on ZSM-5/alumina extrudate particles are described below. In the reference runs, fresh Pt/Ga catalyst charges were loaded “as is,” without any solid diluent. In addition, a charge consisting of 40% v of the Pt/Ga catalyst (specific heat—1.17 kJ/(kg-° K) (0.28 Btu/(lb-° R)) and 60% v of a commercially-available solid inert silica/alumina material (Denstone® 80 ⅛-inch spheres available from Saint-Gobain NorPro; specific heat—1.18 kJ/(kg-° K) (0.28 Btu/(lb-° R)) was tested under the same conditions.

The catalyst used in these tests was prepared on samples of an extrudate material containing 80% wt of CBV 3014E ZSM-5 zeolite (30:1 molar SiO₂:Al₂O₃ ratio; available from Zeolyst International) and 20% wt of alumina binder. This cylindrical extrudate had a diameter of 1.6 mm. The samples were calcined in air up to 425° C. for 1 hr to remove moisture prior to use in catalyst preparation.

Metals were deposited on 100-g samples of the ZSM-5 extrudate by first combining appropriate amounts of stock solutions containing tetraammine platinum nitrate and gallium (III) nitrate, diluting this mixture with deionized water to a volume just sufficient to fill the pores of the extrudate, and impregnating the extrudate with the solution at room temperature and atmospheric pressure. Impregnated samples were aged at room temperature for 2-3 hrs and then dried overnight at 100° C. The target Pt and Ga levels on the catalyst were 0.025% wt and 0.15% wt, respectively.

The catalyst samples described above were tested “as is,” without crushing. Performance tests A, B, and C were conducted with undiluted catalyst. For each of these three tests, a 15-cc charge of catalyst was loaded into a quartz tube (1.40 cm inner diameter) and positioned in a three-zone furnace connected to an automated gas flow system. Performance test D was conducted with catalyst plus solid, inert diluent. For performance test D, the charge consisted of a physical mixture of 6 cc of catalyst plus 9 cc of Denstone® 80 ⅛-inch diameter inert aluminum silicate spheres, available from Saint-Gobain NorPro.

Prior to performance testing, all catalyst charges were pretreated in situ at atmospheric pressure as follows:

(a) calcination with air at 60 L/hr, with reactor wall temperature ramped from 25 to 510° C. in 12 hrs, held at 510° C. for 4-8 hrs, ramped from 510 to 630° C. in 1 hr, and then held at 630° C. for 30 min;

(b) nitrogen purge at 60 L/hr, 630° C. for 20 min;

(c) reduction with hydrogen at 60 L/hr, 630° C. for 30 min.

At the end of the pretreatment, 100% ethane feed was introduced at 1000 GHSV (with respect to catalyst) and atmospheric pressure with the reactor wall temperature maintained at 630° C. The total reactor outlet stream was sampled and analyzed by an online gas chromatography system two minutes after ethane feed addition. Based on the composition data obtained from the gas chromatographic analysis the initial ethane conversion was computed according to the following formula:

ethane conversion,%=100×(100−% wt ethane in outlet stream)/(% wt ethane in feed).

The results of performance tests A, B, C and D, conducted as described above, are presented in Table 2. Average values and standard deviations for the ethane conversion and product selectivities obtained in tests A, B and C are also provided in Table 2. Comparison of the results in Table 2 indicates that 40/60 (v/v) dilution of the catalyst with the Denstone® 80 inert particles did not adversely affect initial activity, benzene yield, or total aromatics yield under the ethane aromatization test conditions used here. Thus, less catalyst produced similar results in test D.

TABLE 2

| | | | | | | |
|---|---|---|---|---|---|---|
| Performance test | A | B | C | Average | Standard | D |
| Amount of | 15 | 15 | 15 | Values, | Deviations, | 6 |
| Catalyst, cc | | | | Tests A-C | Tests A-C | |
| Amount of Inert | 0 | 0 | 0 | | | 9 |
| Diluent, cc | | | | | | |
| Ethane | 46.83 | 46.24 | 46.72 | 46.60 | 0.31 | 47.90 |
| conversion, % | | | | | | |

Reactor Outlet Gas Composition, % wt

| | | | | | | |
|---|---|---|---|---|---|---|
| Hydrogen | 4.16 | 4.14 | 4.10 | 4.13 | 0.03 | 4.26 |
| Methane | 8.20 | 7.80 | 8.31 | 8.10 | 0.27 | 8.50 |
| Ethylene | 5.09 | 5.17 | 5.23 | 5.16 | 0.07 | 5.26 |
| Ethane | 53.17 | 53.76 | 53.28 | 53.40 | 0.31 | 52.10 |
| Propylene | 0.67 | 0.68 | 0.68 | 0.68 | 0.01 | 0.66 |
| Propane | 0.72 | 0.74 | 0.72 | 0.73 | 0.01 | 0.69 |
| C4 | 0.15 | 0.17 | 0.16 | 0.16 | 0.01 | 0.15 |
| C5 | 0.01 | 0.02 | 0.01 | 0.01 | 0.01 | 0.02 |
| Benzene | 15.15 | 14.70 | 15.14 | 15.00 | 0.26 | 14.97 |
| Toluene | 7.91 | 7.96 | 7.97 | 7.95 | 0.03 | 7.67 |
| C8 Aromatics | 1.51 | 1.61 | 1.53 | 1.55 | 0.05 | 1.47 |
| C9+ Aromatics | 3.26 | 3.25 | 2.87 | 3.13 | 0.22 | 4.25 |
| Total Aromatics | 27.83 | 27.51 | 27.51 | 27.62 | 0.18 | 28.36 |

### Example 2

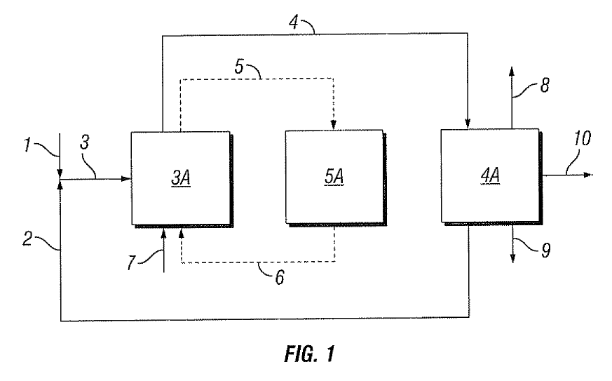

In this example, ethane is converted to aromatic hydrocarbons using the process configuration shown in FIG. 1. 25 tonnes/hr (tph) of stream (1), which primarily constitutes ethane feed (including minor amounts of methane, propane and butane), is mixed with 10 tph of recycle stream (2) that consists primarily of ethane and other hydrocarbons which may include ethylene, propane, propylene, methane, butane and some hydrogen. The total feed amounting to 35 tph (Stream 3) is introduced to the ethane aromatization reactor (3A). The unconverted reactants as well as the products leave the reactor (3A) via stream (4) and are fed to the separation system (4A). The unconverted reactants and light hydrocarbons are recycled back (stream 2) to the reactor while the separation system (4A) yields 7 tph fuel gas (stream 8—predominantly methane and hydrogen), 4 tph C₇₊ liquid products (stream 9) and 13 tph benzene (stream 10).

The aromatization reactor (3A) is a fluidized bed reactor system in which particles of the catalyst used in Example 1 cycle rapidly between a reaction zone where aromatization of the feed takes place and a regeneration zone (5A) where accumulated coke deposits formed on the catalyst surface under aromatization reaction conditions are removed by controlled combustion in an oxygen-containing atmosphere. In this illustrative example, the reactor (3A) operates at about 1 atmosphere pressure and at a temperature range of 590 to 705° C.

The ethane to aromatic conversion process is endothermic and reactor system (3A) requires 73,860 MJ/hr heat energy. In addition, the spent catalyst is prone to deactivation due to coke deposition and must be regenerated subsequently via coke burnoff using mixtures of air or oxygen with nitrogen in the regenerator (5A). The coke burn off step is exothermic liberating about 31,655 MJ/hr of heat energy in the regenerator (5A). This leads to a substantial rise in the temperature causing thermal sintering of the catalyst particles which results in loss in activity. Hence, heat must be removed from the regenerator (5A) using heat exchanger systems (not shown) to limit the temperature rise of the particles to about 675-790° C. Thus, in this example, 17,940 MJ/hr heat (about 60% of the exothermic heat in regenerator 5A) must be removed to prevent the catalyst particles from sintering. 13,715 MJ/hr can be used to heat up the catalyst particles to a temperature above the reactor temperature and transfer the heat to the reactor (3A) without affecting the catalyst performance.

The feed rate of the solid catalyst particles required for the reaction is 180 tph of catalyst circulation rate in this example. Hot catalyst particles act as heat transfer particulate material between the endothermic reactor (3A) and the exothermic regenerator (5A). The modification of this reactor regenerator system (3A), thereby improving the process scheme, is that the solid circulation rate, comprising of the catalyst particles, is increased to 434 tph from 180 tph. The increased flow of catalyst particulates is able to absorb all of the heat liberated from the regenerator (5A) while limiting the temperature increase to reasonable limits to prevent the catalyst from sintering. Thus, the entire 31,655 MJ/hr of heat liberated in the regenerator (5A), which operates at a higher temperature than the reactor, can be transferred to the reactor system (3A). This obviates the need for heat removal system in the regenerator (5A).

In another modification to the system (which is the preferred improvement proposed in this invention), inert solids (such as Denstone®-80 support material) are used as heat transfer particulates and are mixed with the catalyst particles. Denstone®-80 bed support media is described above. These inert particles are significantly less expensive than the catalyst particles but they have the same or similar heat transfer properties, a specific heat of 0.28 Btu/(lb-° R) (1.17 kJ/(kg-° K). The feed rate of the catalyst plus inert particles is kept the same to maintain the same contact time between the feed and the catalyst. This corresponds to 180 tph of catalyst circulation as mentioned earlier. Thus, in this example, a mixture of 180 tph of catalyst and 254 tph of inert solids, totaling to 434 tph of solid mixture, is fed to the reactor system. The solid particulate mixture is able to transfer all the heat in the regenerator (5A) to the reactor (3A) while limiting temperature rise and catalyst sintering. The heat transfer properties of the combined catalyst-Denstone®-80 solid particulate mixture are identical or similar to that of the catalyst particulates. This can be seen from the chemical compositions of the solid particulate system as shown in tables 3, 4 and 5 below (Fr.=fraction).

This mode of operation results in lower catalyst circulation and hence reduction in catalyst losses which are typical of operations involving large solid circulations as observed in fluid catalytic cracking processes.

TABLE 3
Typical chemical composition of the catalyst system

| Catalyst | Wt. Fr. % |
|---|---|
| SiO₂ | 75.7% |
| Al₂O₃ | 24.3% |

TABLE 4
Typical chemical composition of the Denstone ®-80 inerts

| Denstone 80 | wt. fr. % |
|---|---|
| SiO₂ | 66.1 |
| Al₂O₃ | 26.85 |
| TiO₂ | 1.25 |
| K₂O | 2.43 |
| Na₂O | 2.51 |
| CaO | 0.61 |
| MgO | 0.25 |

TABLE 5
Typical chemical composition of catalyst and
Denstone ®-80 inert mixture

| Catalyst-Inert Mixture | wt. fr. (%) |
|---|---|
| SiO₂ | 70.1 |
| Al₂O₃ | 25.8 |
| TiO₂ | 0.7 |
| K₂O | 1.4 |
| Na₂O | 1.5 |
| CaO | 0.4 |
| MgO | 0.1 |

1. A process for producing aromatic hydrocarbons which comprises:
(a) contacting a lower alkane feed with a solid particulate aromatic hydrocarbon conversion catalyst in a fluidized bed reaction zone to produce aromatic hydrocarbons and other products, whereby the catalyst is at least partly deactivated by the formation of undesirable coke deposits,
(b) continuously withdrawing a portion of the catalyst from the reaction zone, regenerating it in a regeneration zone and returning regenerated catalyst to the reaction zone,
(c) maintaining the heat balance between the reaction zone and the regeneration zone by diluting the catalyst particles with particles of a catalytically inactive solid with about the same or improved specific heat relative to the catalyst,
(d) separating aromatic hydrocarbons from the other products and unreacted lower alkanes, and
(e) optionally recycling unreacted lower alkanes to the reaction zone.

2. The process of claim 1 wherein the ratio of the circulation rate of the catalytically inactive solid to the circulation rate of the catalyst particles is from 1:6 to 6:1.

3. The process of claim 2 wherein the ratio of the circulation rate of the catalytically inactive solid to the circulation rate of the catalyst particles is from 0.4:1 to 2.5:1.

4. The process of claim 1 wherein the specific heat of the catalytically inactive solid is at least 0.2 Btu/(lb-° R) (0.8 kJ/(kg-° K)) at the temperature of operation.

5. The process of claim 4 wherein the specific heat of the catalytically inactive solid is from 0.2 to 0.4 Btu/(lb-° R)(0.8 to 1.7 kJ/(kg-° K)) at the temperature of operation.

6. The process of claim 5 wherein the specific heat of the catalytically inactive solid is from 1.04 to 1.5 kJ/(kg-° K).
